(19) United States
(12) Patent Application Publication
(10) Pub. No.: US 2021/0133556 A1
(43) Pub. Date: May 6, 2021

(54) FEATURE-SEPARATED NEURAL NETWORK PROCESSING OF TABULAR DATA

(71) Applicant: INTERNATIONAL BUSINESS MACHINES CORPORATION, Armonk, NY (US)

(72) Inventors: Toshiya Iwamori, Tokyo (JP); Akira Koseki, Kanagawa-ken (JP)

(21) Appl. No.: 16/669,641

(22) Filed: Oct. 31, 2019

(51) Int. Cl. G06N 3/08; G06F 16/28; G06N 3/04; G16H 10/60; G06Q 30/02

(52) U.S. Cl. CPC G06N 3/08; G06F 16/285; G06Q 30/0246; G06N 3/0481; G16H 10/60; G06N 3/04

(57) ABSTRACT

Methods and systems for classifying tabular data include clustering columns from one or more input tables into column groups. The column groups are processed using a neural network that has a set of input layers, each input layer accepting a respective one column group from the column groups as input, to generate a classification output. A classification task is performed on the one or more input tables using the classification output.

### BACKGROUND

The present invention generally relates to neural networks and, more particularly, to the use of neural networks to process tabular data.

An artificial neural network (ANN) is an information processing system that is inspired by biological nervous systems, such as the brain. The key element of ANNs is the structure of the information processing system, which includes a large number of highly interconnected processing elements (called “neurons”) working in parallel to solve specific problems. ANNs are furthermore trained in-use, with learning that involves adjustments to weights that exist between the neurons. An ANN is configured for a specific application, such as pattern recognition or data classification, through such a learning process.

Referring now to FIG. 1, a generalized diagram of a neural network is shown. ANNs demonstrate an ability to derive meaning from complicated or imprecise data and can be used to extract patterns and detect trends that are too complex to be detected by humans or other computer-based systems. The structure of a neural network is known generally to have input neurons 102 that provide information to one or more “hidden” neurons 104. Connections 108 between the input neurons 102 and hidden neurons 104 are weighted and these weighted inputs are then processed by the hidden neurons 104 according to some function in the hidden neurons 104, with weighted connections 108 between the layers. There can be any number of layers of hidden neurons 104, and as well as neurons that perform different functions. There exist different neural network structures as well, such as feed forward neural network, convolutional network, etc. Finally, a set of output neurons 106 accepts and processes weighted input from the last set of hidden neurons 104.

This represents a “feed-forward” computation, where information propagates from input neurons 102 to the output neurons 106. Upon completion of a feed-forward computation, the output is compared to a desired output available from training data. The error relative to the training data is then processed in “feed-back” computation, where the hidden neurons 104 and input neurons 102 receive information regarding the error propagating backward from the output neurons 106. Once the backward error propagation has been completed, weight updates are performed, with the weighted connections 108 being updated to account for the received error. This represents just one variety of ANN.

While ANNs are suitable for a wide variety of tasks, certain ANN structures are more appropriate for particular kinds of input data. For example, convolutional neural networks (CNNs) are effective for handling two-dimensional image data. Using an ANN on tabular data, however, is challenging.

### SUMMARY

A method for classifying tabular data includes clustering columns from one or more input tables into column groups. The column groups are processed using a neural network that has a set of input layers, each input layer accepting a respective one column group from the column groups as input, to generate a classification output. A classification task is performed on the one or more input tables using the classification output.

A system for classifying tabular data includes a hardware processor and a memory, coupled to the hardware processor, configured to store a column clusterer that, when executed by the hardware processor, clusters columns from one or more input tables into a plurality of column groups, and to further store a classification module that, when executed by the hardware processor, performs a classification task on the one or more input tables using a classification output. An artificial neural network is configured to process the plurality of column groups and has a plurality of input layers, each input layer accepting a respective one column group from the plurality of column groups as input, to generate the classification output.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a prior art diagram of a neural network structure;

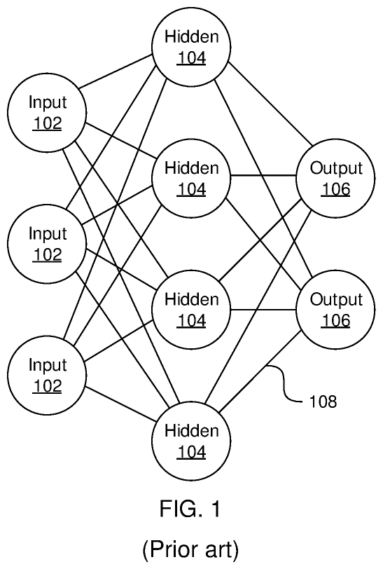

FIG. 2 is a diagram of tabular data which includes multiple sets of correlated columns, where the correlated columns are not necessarily near one another in the table, in accordance with an embodiment of the present invention;

FIG. 3 is a block/flow diagram of performing a classification task on tabular data by clustering correlated columns in an input table in accordance with an embodiment of the present invention;

FIG. 4 is a block diagram of a neural network structure that accepts clustered input table columns in respective input layers to preserve features of low-impact columns in densely connected neural network layers in accordance with an embodiment of the present invention;

FIG. 5 is a diagram of an exemplary neural network architecture that can be used to implement the neural network structure in accordance with an embodiment of the present invention;

FIG. 6 is a block diagram of a tabular data processing system that accepts tabular data as an input and clusters the data according to correlated columns to improve accuracy in a classification task in accordance with an embodiment of the present invention; and

FIG. 7 is a block diagram of a processing system suitable to implement the tabular data processing system in accordance with an embodiment of the present invention.

### DETAILED DESCRIPTION

Embodiments of the present invention provide a machine learning system that processes tabular data in a manner that captures the representations of less significant features. Because CNNs are sensitive to correlations between neighboring entries in a two-dimensional input, tabular data that lacks such correlations can be a challenging type of input. For example, the position of a particular column (e.g., whether it is the first column, the second column, and so on) generally carries no informational value. This contrasts to an image, where the values of a column are can be closely correlated to the values of its neighboring columns. Thus, a CNN, which accepts the input in a space-dependent manner, will not be able to capture any correlations that may exist between columns that are positioned far apart.

Because of this, tabular data can be classified using fully connected neural network architectures. Such neural networks learn hidden representations from all of the input features at once, so that feature clusters can be learned and so that features in a more significant cluster are more heavily weighted than features in a less significant cluster. Such networks have difficulty learning effective representations from the features in the less significant clusters.

To address this problem, the present embodiments pre-cluster columns according to their correlations. By splitting the input according to their correlations, the present embodiments split the input features to the clusters, thereby preserving less significant features. Each cluster of columns is then used as input separately to a neural network layer, the outputs of which are concatenated and used as input to a classifier. Each cluster's contributions are thereby captured and maintained through subsequent classification steps. Clustering the input data according to correlations improves the classifier without applying domain knowledge, making the present embodiments applicable to any kind of tabular data. The present embodiments improve the accuracy of classification tasks by preserving the contributions of columns that have relatively low impact on the result and that would otherwise be wiped out in a densely connected input layer.

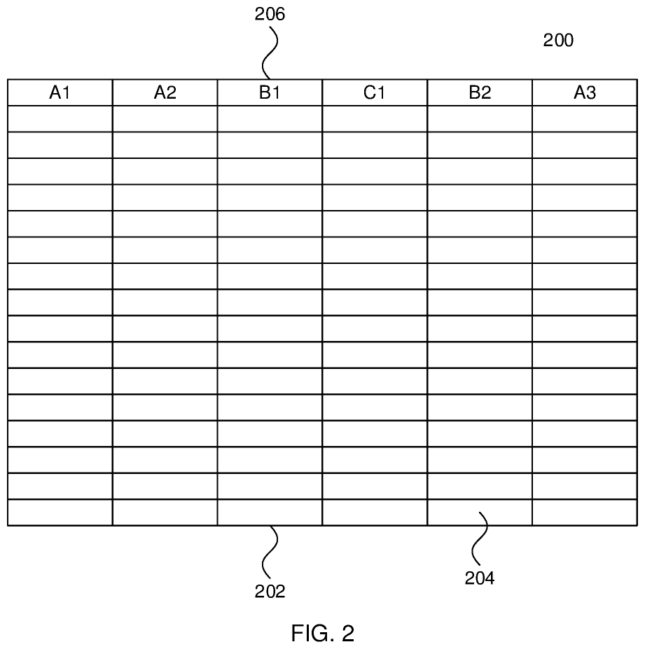

Referring now to the drawings in which like numerals represent the same or similar elements and initially to FIG. 2, a diagram of a set of tabular data 200 is shown. The tabular data 200 is organized into vertical columns 202 that include data elements 204. The columns are depicted as belonging to groups 206, designated herein as A, B, and C. These designations do not necessarily represent explicit data values stored in the data elements 204, but are instead used herein to show that the columns 202 that share a group designation have correlations between their respective data elements 204.

As can be seen, while some columns 202 from a give group 206 (e.g., group A) may be located close to one another in the table 200, other members of the group 206 may be separated, with columns 202 from other groups 206 in between. The present embodiments identify the groups using a measure of correlation between the data elements of respective columns and then clusters columns 202 according to the identified groups.

The clustered groups are treated as separate tables and are processed separately to identify the features of each group. This ensures that the features of every group 206 is represented, even though some groups 206 may have a relatively small impact on the ultimate classification. While these groups have a small impact, inclusion of their features in the classification input improves the accuracy of the classification output.

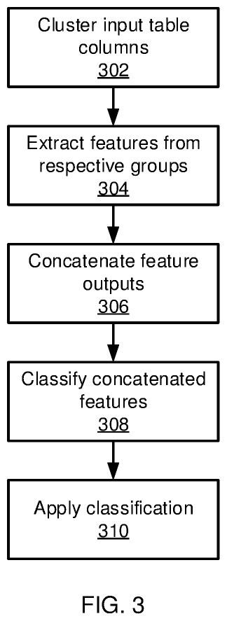

Referring now to FIG. 3, a method of classifying tabular data is shown. Block 302 clusters the input table columns 202 according to correlations between the data elements 204 in the respective columns 202. Correlations can be determined using a correlation metric to produce a correlation matrix that identifies, for each pair of columns, a correlation value, with higher correlation values indicating a greater amount of correlation between the respective columns. Any appropriate correlation metric can be used, for example generating outputs with values between zero and one. In this example, a value of zero would indicate no correlation between the pair of columns, while a value of one would indicate that the columns are exactly the same. This clustering can be performed using, e.g., a k-means clustering process that groups the n different columns 202 into k groups 206. The parameter k can be set by a user or can, alternatively, be set automatically according to a determination of a number of clusters that takes into account some variance measure within the clusters. Thus, columns that have a greater measurement of correlation among them will be clustered together.

Block 304 extracts features from the respective groups 206. In some embodiments, block 304 uses a neural network layer formed from, e.g., a fully connected neural network layer with batch normalization and dropout functions. Block 306 then concatenates the outputs from the respective groups 206 to form a single feature vector. Block 308 then classifies the concatenated features using, e.g., a set of additional fully connected layers. Additional detail regarding the structure of the neural network will be described below.

Once classification is complete, block 310 applies the classification to a practical purpose. For example, tabular data classification can be used to process medical records of a patient, where a large volume of data can be quickly assessed to determine whether it indicates a particular medical condition, leading to rapid diagnosis and treatment. In another example, tabular data can be classified to help predict click-throughs for online advertisements. In any application, the present embodiments for tabular data classification provide superior accuracy in the outcome.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), FPGAs, and/or PLAs.

These and other variations of a hardware processor subsystem are also contemplated in accordance with embodiments of the present invention.

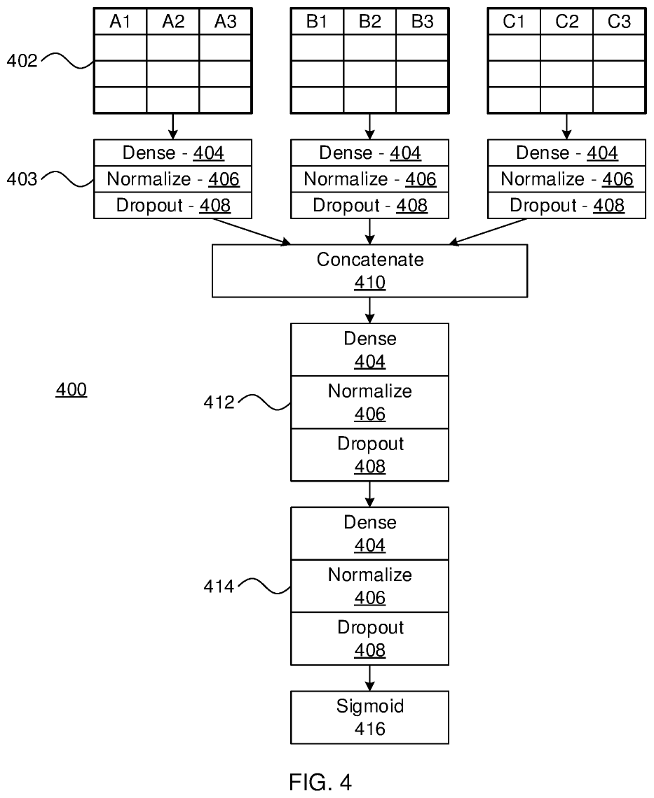

Referring now to FIG. 4, a high-level diagram of a neural network structure is shown. The network 400 takes as input a set of table groups 402. Each group includes one or more columns that were found to be correlated to one another in block 302 above. The groups 402 are input as tabular data to respective first layers 403. Each of the first layers 403 include a densely connected (i.e., fully connected) neural network layer 404, a batch normalization function 406, and a dropout function 408. It should be understood that there can be any appropriate number of first layers 403, each accepting a respective table group 402 as input. The number of table groups 402 can be selected to correspond to the number of first layers 403 by setting a number of clusters in block 302.

The outputs of the first layers 403 are concatenated at block 410. For example, if there are eight first layers 403, and each outputs a feature vector having 128 dimensions, then concatenation can generate a feature vector having 128*K dimensions, with K representing the number of clusters. This concatenated vector is used as an input to a second layer 412, which also includes a densely connected neural network layer 404, a batch normalization function 406, and a dropout function 408 to prevent overfitting. For example, the second layer 412 can accept a vector having 1024 dimensions and output a vector having 512 dimensions.

Any number of additional layers 414 can be used to bring the dimensionality down to a predetermined size. Following the example above, additional layers can be used to bring the dimensionality of the feature vector down to 64. The final feature vector is processed by a sigmoid layer 416 to produce an output between zero and one. In one specific embodiment, there can be twelve layers in total, including an input layer, a clustering layer, a dense layer that uses ReLU activation for each table group 402 (having parameters of <input dimensionality>, 128), a batch normalization layer of each table group 402 (having parameters 128, 128), a dropout layer for each group (128, 128), a dense layer with ReLU activation (128*K, 512), a batch normalization layer (512, 512), a dropout layer (512, 512), a dense layer with ReLU activation (512, 64), a batch normalization layer (64, 64), a dropout layer (64, 64), a dense layer with sigmoid activation (64,1).

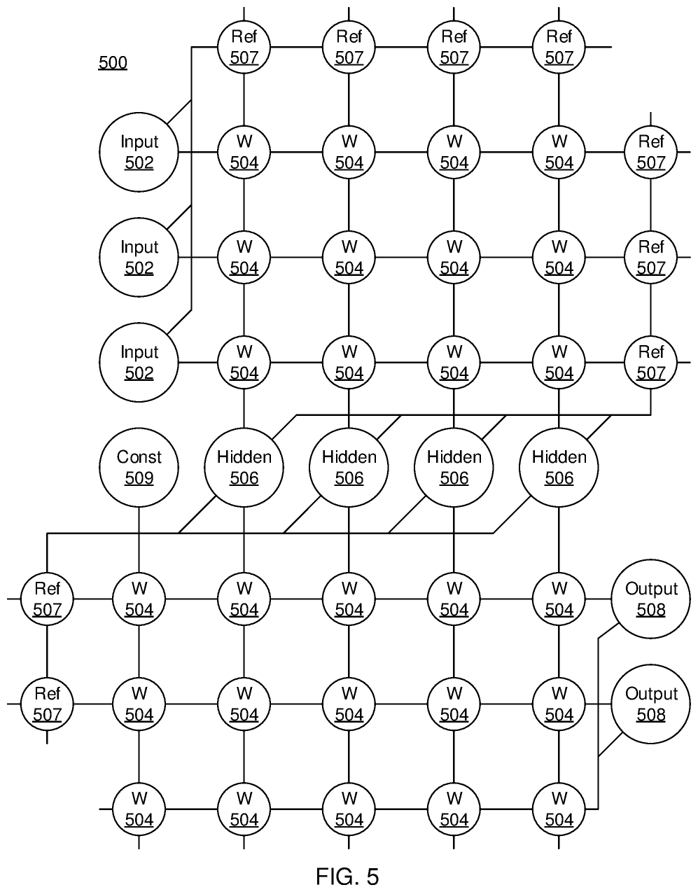

Referring now to FIG. 5, an artificial neural network (ANN) architecture 500 is shown. It should be understood that the present architecture is purely exemplary and that other architectures or types of neural network can be used instead. In particular, while a hardware embodiment of an ANN is described herein, it should be understood that neural network architectures can be implemented or simulated in software. The hardware embodiment described herein is included with the intent of illustrating general principles of neural network computation at a high level of generality and should not be construed as limiting in any way.

Furthermore, the layers of neurons described below and the weights connecting them are described in a general manner and can be replaced by any type of neural network layers with any appropriate degree or type of interconnectivity. For example, layers can include convolutional layers, pooling layers, fully connected layers, softmax layers, or any other appropriate type of neural network layer. Furthermore, layers can be added or removed as needed and the weights can be omitted for more complicated forms of interconnection.

During feed-forward operation, a set of input neurons 502 each provide an input voltage in parallel to a respective row of weights 504. In the hardware embodiment described herein, the weights 504 each have a settable resistance value, such that a current output flows from the weight 504 to a respective hidden neuron 506 to represent the weighted input. In software embodiments, the weights 504 can simply be represented as coefficient values that are multiplied against the relevant neuron outputs.

Following the hardware embodiment, the current output by a given weight 504 is determined as

$$I = \frac{V}{r} ,$$

where V is the input voltage from the input neuron 502 and r is the set resistance of the weight 504. The current from each weight adds column-wise and flows to a hidden neuron 506. A set of reference weights 507 have a fixed resistance and combine their outputs into a reference current that is provided to each of the hidden neurons 506. Because conductance values can only be positive numbers, some reference conductance is needed to encode both positive and negative values in the matrix. The currents produced by the weights 504 are continuously valued and positive, and therefore the reference weights 507 are used to provide a reference current, above which currents are considered to have positive values and below which currents are considered to have negative values. The use of reference weights 507 is not needed in software embodiments, where the values of outputs and weights can be precisely and directly obtained. As an alternative to using the reference weights 507, another embodiment can use separate arrays of weights 504 to capture negative values.

The hidden neurons 506 use the currents from the array of weights 504 and the reference weights 507 to perform some calculation. The hidden neurons 506 then output a voltage of their own to another array of weights 504. This array performs in the same way, with a column of weights 504 receiving a voltage from their respective hidden neuron 506 to produce a weighted current output that adds row-wise and is provided to the output neuron 508.

It should be understood that any number of these stages can be implemented, by interposing additional layers of arrays and hidden neurons 506. It should also be noted that some neurons can be constant neurons 509, which provide a constant output to the array. The constant neurons 509 can be present among the input neurons 502 and/or hidden neurons 506 and are only used during feed-forward operation.

During back propagation, the output neurons 508 provide a voltage back across the array of weights 504. The output layer compares the generated network response to training data and computes an error. The error is applied to the array as a voltage pulse, where the height and/or duration of the pulse is modulated proportional to the error value. In this example, a row of weights 504 receives a voltage from a respective output neuron 508 in parallel and converts that voltage into a current which adds column-wise to provide an input to hidden neurons 506. The hidden neurons 506 combine the weighted feedback signal with a derivative of its feed-forward calculation and stores an error value before outputting a feedback signal voltage to its respective column of weights 504. This back propagation travels through the entire network 500 until all hidden neurons 506 and the input neurons 502 have stored an error value.

During weight updates, the input neurons 502 and hidden neurons 506 apply a first weight update voltage forward and the output neurons 508 and hidden neurons 506 apply a second weight update voltage backward through the network 500. The combinations of these voltages create a state change within each weight 504, causing the weight 504 to take on a new resistance value. In this manner the weights 504 can be trained to adapt the neural network 500 to errors in its processing. It should be noted that the three modes of operation, feed forward, back propagation, and weight update, do not overlap with one another.

As noted above, the weights 504 can be implemented in software or in hardware, for example using relatively complicated weighting circuitry or using resistive cross point devices. Such resistive devices can have switching characteristics that have a non-linearity that can be used for processing data. The weights 504 can belong to a class of device called a resistive processing unit (RPU), because their non-linear characteristics are used to perform calculations in the neural network 500. The RPU devices can be implemented with resistive random access memory (RRAM), phase change memory (PCM), programmable metallization cell (PMC) memory, or any other device that has non-linear resistive switching characteristics. Such RPU devices can also be considered as memristive systems.

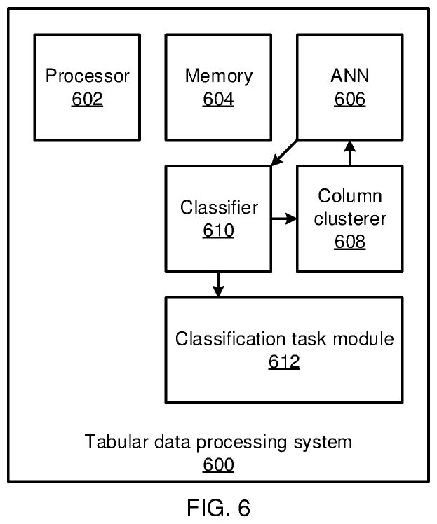

Referring now to FIG. 6, a tabular data processing system 600 is shown. The system 600 includes a hardware processor 602 and a memory 604. An ANN 606 is implemented in, for example, software that is stored in the memory 604 and that is executed by the hardware processor 602. In other embodiments, the ANN 606 can be implemented using dedicated hardware components. In yet other embodiments, the ANN 606 can be implemented in a combination of hardware and software. It is specifically contemplated that the ANN 606 can have a structure similar to that described above, with multiple input layers and one or more subsequent layers, but it should be understood that any appropriate neural network structure can be used instead.

A classifier 610 receives a set of tabular data as input. This input may include one or more tables arranged, for example, as a matrix of columns and rows. A column clusterer 608 accepts the input table(s) and clusters the columns into k groups. These groups are each used as an input to a respective input layer of the ANN 606. The ANN 606 generates an output that the classifier 610 uses to determine an outcome. For example, in a medical data embodiment, where the tabular input represents a patient's medical information and may include, for example, measurements of the patient's condition over time, the classifier 610 can use the ANN 606 to identify labels for conditions that the patient may have. Because the columns were clustered before being passed to the ANN 606, features that might otherwise have been drowned out in a fully connected layer are preserved. The contribution of these features improves the classification's accuracy.

A classification task module 612, which can be implemented as software that is stored in memory 604 and can be executed by the hardware processor 602, executes a classification task with the input tables, based on the classifier output. For example, the classification task module 612 can perform click-through prediction for online advertisements.

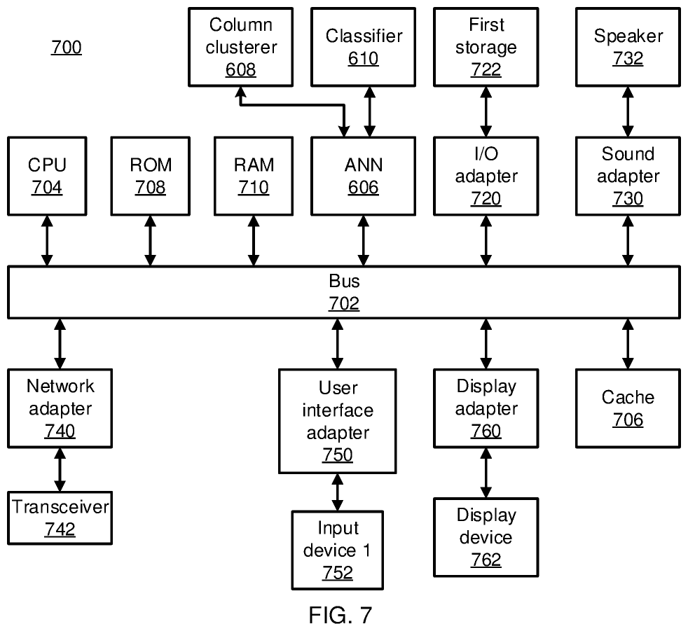

Referring now to FIG. 7, an exemplary processing system 700 is shown which may represent the tabular data processing system 600. The processing system 700 includes at least one processor (CPU) 704 operatively coupled to other components via a system bus 702. A cache 706, a Read Only Memory (ROM) 708, a Random Access Memory (RAM) 710, an input/output (I/O) adapter 720, a sound adapter 730, a network adapter 740, a user interface adapter 750, and a display adapter 760, are operatively coupled to the system bus 702.

A first storage device 722 is operatively coupled to system bus 702 by the I/O adapter 720. The storage device 722 can be any of a disk storage device (e.g., a magnetic or optical disk storage device), a solid state magnetic device, and so forth. The storage device 722 can be the same type of storage device or different types of storage devices.

A speaker 732 is operatively coupled to system bus 702 by the sound adapter 730. A transceiver 742 is operatively coupled to system bus 702 by network adapter 740. A display device 762 is operatively coupled to system bus 702 by display adapter 760.

A first user input device 752 is operatively coupled to system bus 702 by user interface adapter 750. The user input device 752 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input device 722 can be the same type of user input device or different types of user input devices. The user input device 752 is used to input and output information to and from system 700.

Of course, the processing system 700 may also include other elements (not shown), as readily contemplated by one of skill in the art, as well as omit certain elements. For example, various other input devices and/or output devices can be included in processing system 700, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 700 are readily contemplated by one of ordinary skill in the art given the teachings of the present principles provided herein.

Having described preferred embodiments of feature-separated neural network processing tabular data (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for classifying tabular data, comprising:
clustering a plurality of columns from one or more input tables into a plurality of column groups;
processing the plurality of column groups using a neural network that has a plurality of input layers, each input layer accepting a respective one column group from the plurality of column groups as input, to generate a classification output; and
performing a classification task on the one or more input tables using the classification output.

2. The method of claim 1, wherein processing the plurality of column groups further comprises concatenating respective outputs of the plurality of input layers into a single feature vector.

3. The method of claim 1, wherein each input layer includes a respective densely connected layer.

4. The method of claim 3, wherein each input layer further includes a respective batch normalization function and a respective dropout function that operate on an output of the respective densely connected layer.

5. The method of claim 1, wherein the neural network further includes one or more hidden layers that process the outputs of the input layers and that each include a respective densely connected layer.

6. The method of claim 1, wherein processing the plurality of column groups in separate input layers preserves contributions from columns that would be lost if the plurality of column groups were processed by a single densely connected layer.

7. The method of claim 1, wherein clustering the columns includes generating a correlation matrix that identifies a correlation value for each pair of the columns.

8. The method of claim 7, wherein clustering the columns is performed using a k-means clustering process.

9. The method of claim 1, wherein the neural network further includes a sigmoid output layer that generates the classification output.

10. The method of claim 1, wherein the classification task is selected from a group consisting of click-through prediction for advertisements and diagnosis and treatment of a patient's health condition.

11. A non-transitory computer readable storage medium comprising a computer readable program for classifying tabular data, wherein the computer readable program when executed on a computer causes the computer to perform the steps of:
clustering a plurality of columns from one or more input tables into a plurality of column groups;
processing the plurality of column groups using a neural network that has a plurality of input layers, each input layer accepting a respective one column group from the plurality of column groups as input, to generate a classification output; and
performing a classification task on the one or more input tables using the classification output.

12. A system for classifying tabular data, comprising:
a hardware processor;
a memory, coupled to the hardware processor, configured to store a column clusterer that, when executed by the hardware processor, clusters a plurality of columns from one or more input tables into a plurality of column groups, and to further store a classification module that, when executed by the hardware processor, performs a classification task on the one or more input tables using a classification output; and
an artificial neural network, configured to process the plurality of column groups, that has a plurality of input layers, each input layer accepting a respective one column group from the plurality of column groups as input, to generate the classification output.

13. The system of claim 12, wherein the artificial neural network is further configured to concatenate respective outputs of the plurality of input layers into a single feature vector.

14. The system of claim 12, wherein each input layer includes a respective densely connected layer.

15. The system of claim 14, wherein each input layer further includes a respective batch normalization function and a respective dropout function that operate on an output of the respective densely connected layer.

16. The system of claim 12, wherein the neural network further includes one or more hidden layers that process the outputs of the input layers and that each include a respective densely connected layer.

17. The system of claim 12, wherein the artificial neural network is configured to preserve contributions from columns that would be lost if the plurality of column groups were processed by a single densely connected layer.

18. The system of claim 12, wherein the column clusterer is further configured to generate a correlation matrix that identifies a correlation value for each pair of the columns.

19. The system of claim 18, wherein the column clusterer is further configured to cluster the columns according to a k-means clustering process.

20. The system of claim 12, wherein the artificial neural network further includes a sigmoid output layer that generates the classification output.
